(19) United States
(12) Patent Application Publication
(10) Pub. No.: US 2006/0069387 A1
(43) Pub. Date: Mar. 30, 2006

(54) Comprehensive system that minimizes outbreak of operating room fires

(76) Inventor: Tewodros Gedebou, Los Angeles, CA (US)

Correspondence Address: STETINA BRUNDA GARRED & BRUCKER, 75 ENTERPRISE, SUITE 250, ALISO VIEJO, CA, 92656, US

(21) Appl. No.: 10/949,856

(22) Filed: Sep. 24, 2004

(51) Int. Cl. A61B 18/14; A61B 18/04; A61B 18/20

(52) U.S. Cl. 606/45; 606/49; 606/42; 606/13; 606/28

(57) ABSTRACT

A system for prevention of fires in operating rooms as frequently arises from electrocautery and laser systems comprising an electrocautery instrument having a shielding gas that is expelled from the distal-most end thereof to thus prevent the cautery tip spark from coming into contact with an oxygen-enriched environment that may otherwise propagate the spark into a full-fledged fire. The systems can further be coupled with oxygen sensors, alarms and mechanisms for limiting the delivery of oxygen. Additional refinements include incorporating heat sensory strips to the distal ends of oxygen delivery systems, which are in turn coupled with a thermocouple device set to turn off oxygen delivery or electrosurgical system at given levels of heat to thereby limit the extent of burn injury. Further refinements also include the use of nonflammable inserts to the tips of the oxygen delivery systems such that in the event of a fire the plastic does not catch on fire and in effect turn into a blowtorch.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND OF THE INVENTION

Operating room fires and the hazards associated therewith are well known in the art. Although there have been multiple reports over the past few decades, hundreds of operating room fires continue to occur annually during the performance of a variety of surgical procedures. Although relatively infrequent, such patient fires result in dramatic burn injury as well as patient fatality when they occur. Serious injury to surgeons and other health care workers also frequently occurs, as does substantial property damage to the operating room facility.

The three ingredients of fire, defined as rapid exothermic reaction, include an ignition source, an oxidizer and fuel. Fuels include a wide variety of materials such as operating room gowns, surgical drapes, various prepping agents, patient hair, plastic respiratory equipment and the like. With respect to the ignition source, it is well known that a variety of surgical equipment, and in particular electrocautery surgical instruments and lasers are known to emit substantial heat. Moreover, the tip of the electrocautery knife, due to the electrical current passing through, or the beam of the laser device is exceptionally prone to ignite a fire. A high concentration of oxygen and other flammable gases such as nitrous oxide are also typically present during surgery, particularly during surgical procedures involving the head and neck insofar as oxygen and nitrous oxide tend to build beneath the surgical drapes or in the oropharyngeal cavity, which thus are operative to create a highly combustible atmosphere. In such an oxygen-enriched environment, materials that are not considered flammable in normal circumstances such as surgical drapes or respiratory apparatus can easily ignite with the resultant fire burning more violently and/or at higher temperatures. In the case of the latter, the respiratory system consisting of various plastics at the distal ends catches on fire and with the blowing oxygen simulates a blowtorch significantly worsening the burn injury. A multitude of specific cases have been reported as well such as explosion of bowel being cauterized, lung surgery, laser damage to respiratory tubes, ignition of pooled prepping fluid, etc.

Despite the well-known hazards associated with performing surgery under such conditions, however, there has not heretofore been any effective type of system or method that is operative to minimize the potential outbreak of operating room fires. In this regard, the best safety practices currently in use merely involve taking precautionary measures and typically consist of nothing more than making efforts to minimize the build up of oxygen and nitrous oxide, activating electrosurgical and electrocautery units at lower power settings, and/or only using such instruments when the cautery tips thereof are within view. Additional precautions include turning equipment off when not in use or otherwise placing electrosurgical instruments in a safe location, such as a safety holster, when not in active use. Likewise recommended is the practice of allowing a certain amount of time, like a minute or more, to discontinue oxygen administration to the patient prior to the use of the electrosurgical instruments, lasers and the like.

Notwithstanding such safeguards, even the best practices are not effective to substantially reduce the risk of operating room fires. In this regard, there is simply no system or method currently available that enables high-risk surgical equipment, and in particular electrosurgical instruments such as electrocautery pin knives and the like, or lasers to be effectively utilized in oxygen-enriched environments while at the same time effectively eliminate the potential for such elements to create a fire hazard. There is likewise substantially lacking in the art any type of system and method for reducing the risk of operating room fires that can be readily integrated as part of an existing electrosurgical device, and in particular an electrocautery apparatus that can be utilized per conventional electrocautery instruments and be utilized per conventional electrosurgical instruments for use in performing a wide variety of surgical procedures. There is likewise a need for such a system and method that is of simple construction, exceptionally low cost, very safe to utilize and can be constructed utilizing well-known, commercially available materials.

### BRIEF SUMMARY OF THE INVENTION

The main embodiment of the invention includes an electrocautery instrument or laser device that will be operatively coupled to a source of inert gas, such as nitrogen, helium, air, argon, carbon dioxide, or other non-toxic gaseous flame retardant such as halon, that will be fluidly coupled to the electrocautery element and operative to be dispersed through the distal-most end, thereof. According to such embodiment, the source of inert gas will be coupled to the electrocautery instrument such that the inert gas is expelled from the distal-most end, either by automatic or manually operable control, and preferably radially about the electrocautery blade or laser point utilized to perform the surgical procedure. To that end, it is contemplated that such inert gas, which may be maintained at either ambient temperature or otherwise cooled, may be continuously free flowing through the electrocautery instrument throughout the surgical procedure, or may be coupled to a switch, such as a two step switch, to the extent that the gas flow is instigated first before the electrocautery. In the latter case, turning off the electrocautery instrument would similarly require the gas flow to continue thereafter thereby assuring that the electrocautery process is fully shielded by inert gas flow. Alternatively, the switch activating the electrocautery instrument may be coupled with a sensor located within the inert gas delivery tube that allows activation of the electrocautery instrument only after the flow of inert gas is established (i.e., operation of device is permitted once the flow of inert gas reaches a pre-determined level. In this regard, such inert gas will be operative to surround the environment about the distal-most end where the electrocautery blade is utilized to thus prevent any heat or spark generated thereby from coming into contact with the oxygen-enriched environment by blowing away the oxygen or other flammable gases. Under such circumstances, the electrocautery instrument will be incapable of igniting an operating room fire that may otherwise spread widely. Similarly, it is contemplated that a laser instrument as coupled with the inert source of gas may be operative such that the inert gas is to be distributed from the distal-most end of the laser instrument prior to when the laser beam of the instrument is turned on or applied to tissue, or any other type of ignitable substance.

An additional object of the present invention includes a cooling mechanism to the gas as is well-known by those familiar with the art, so as to limit the burn injury caused by the laser or electrocautery instrument to the point of contact only.

In another embodiment of this comprehensive system is the addition of a heat sensor strip, such as thermister or thermocouple device, operatively attached to the distal ends of a ventilator apparatus utilized in conjunction when the surgical procedure is performed, that is coupled with an oxygen release valve and/or the electrocautery device such that the apparatus automatically turns off oxygen delivery and/or electrosurgical device in the event of reaching predetermined temperature level. An added feature could include setting off an alarm when a given temperature level is reached so as to warn the anesthesiologist from continuing the flow of oxygen or nitrous oxide, as well as to warn the surgeon to refrain from using energy transmission from the electrosurgical instrument. Moreover, it is contemplated that the heat sensor device can be operatively coupled with the inert gas flushing mechanism and thus designed to be automatically turned on when preset temperature levels are recorded.

In a further embodiment of this system, it is contemplated that the distal most part of the ventilation system will be provided with nonflammable materials such as Teflon, metals and the like so as to prevent the respiratory system from catching on fire and turning into a blowtorch.

### BRIEF DESCRIPTION OF THE DRAWINGS

These as well as other features of the present invention will become more apparent upon reference to the drawings.

FIG. 1 is a schematic view of an electrosurgical system for performing electrocautery surgical procedures that substantially reduces the possibility for such system to ignite or otherwise cause an operating room fire.

FIG. 2 is a cross-sectional view of an electrocautery surgical instrument constructed in accordance with the preferred embodiment of the present invention.

FIG. 3 is a perspective view, shown partially in cross-section, of a ventilation system for use in combination with an electrocautery surgical system that further substantially reduces the possibility for the electrocautery system to ignite or otherwise cause an operating room fire.

### DETAILED DESCRIPTION OF THE INVENTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

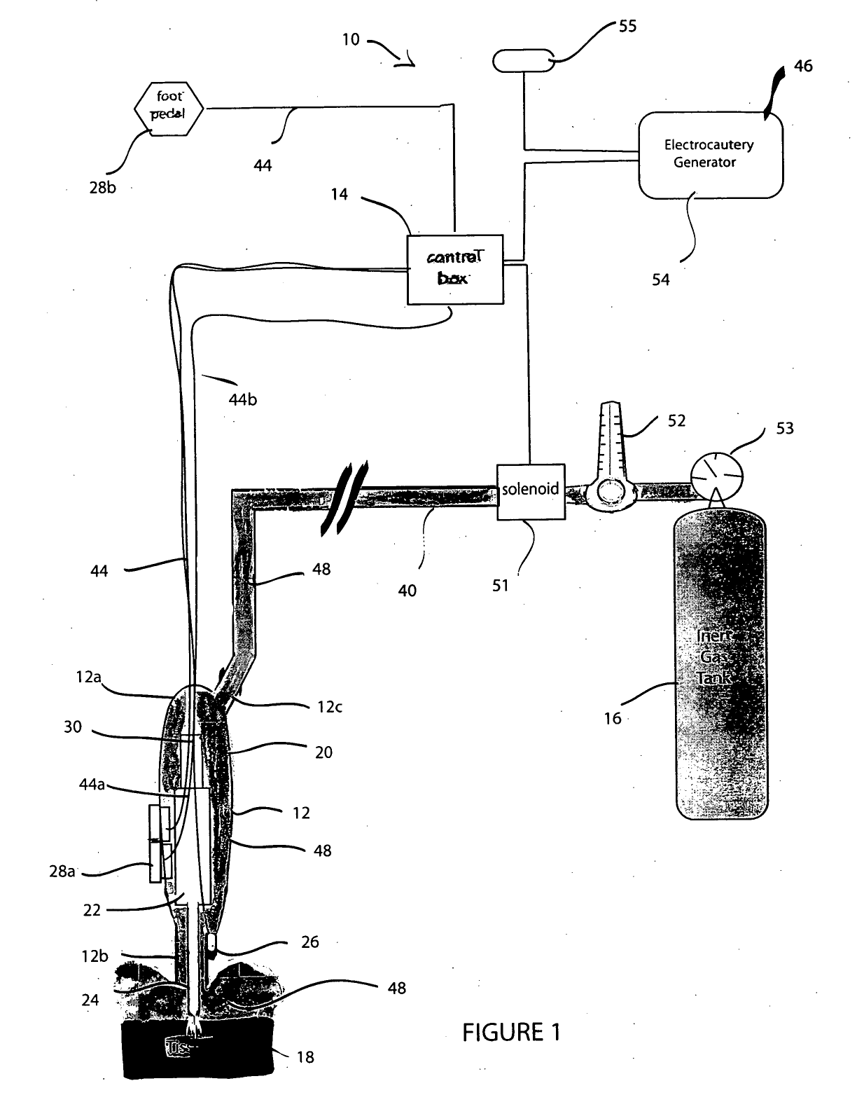

Referring now to the figures, and initially to FIG. 1, there is shown an electrosurgical system 10 for performing electrocautery surgical procedures that substantially reduces, if not eliminates, the possibility for such system 10 to ignite or otherwise cause and operating room fire. As shown, the system 10 comprises three essential components, namely, a hand-held electrocautery instrument 12, a control unit 14, and a supply of inert gas 16, which may comprise nitrogen, helium, carbon dioxide, air, argon or any other type of gas known to be generally non-reactive or can serve as a non-toxic flame retardant, such as halon. With respect to the latter, it should be understood that any non-toxic flame retardant should be considered to fall within the scope of inert gas as used herein.

With respect to the former, the electrocautery instrument 12, as per conventional electrocautery instruments, is preferably formed as an elongate pen-knife having a proximal end 12a, which is fluidly coupled to the gas supply 16 via tubing 40, discussed more fully below, and a distal end 12b, which is oriented toward the surgical site to which the electrocautery instrument is utilized, such as to coagulate bleeding vessels or cut through tissue 18. Electrocautery device 12 thus defines a housing having an interior 20, within which is an electrocautery element 22. Formed on the distal-most end of such cutting/coagulating (cautery) element 22 is a cautery tip 24 that is operative to be extended from the distal-most end instrument 12 to thus enable the same to cut through a given site of tissue. To achieve that end, the cautery element 22 is coupled to a power source via a connection 44a, the latter extending via cord 44 to control unit 14, to the electrocautery generator 54 and ultimately an external power source provided at 46. As per conventional electrocautery devices, electrocautery device 12 is provided with a manually 28a (or by foot 28b) operable switch that is electrically coupled via link 30 to the power connection 44a to thus selectively actuate the cautery element 22, and in particular cautery tip 24 thereof. Upon activation of the switch 28, a signal will be transmitted via link 44 to control unit 14 which in turn would activate a solenoid valve 51 to cause gas contained within gas supply 16 to be emitted through valve 42, preset at a given flow rate determined by a connected flow meter 52, and via line 40 into the interior 20 of the electrocautery device 12 via duct 12c. In this regard, such inert gas, represented by 48, will be operative to flow through and outwardly from the distal end 12b of the electrocautery device 12 such that the cautery tip 24 becomes immersed in a flow of inert gas 48.

As a consequence, the surrounding air containing oxygen or any other flammable gas will be blown away from the cautery tip 24 and thus incapable of being ignited by any spark. In further refinements of the invention, it is contemplated that inert gas 48 may be designed to be continuously free flowing through the electrocautery device 12 such that the cautery tip 24 thereof is constantly receiving an outflowing source of inert gas 48 engulfed thereabout. Along these lines, it is contemplated that the system 10 can be engineered such that the inert gas 48 is caused to flow through the distal end 12b of the electrocautery device 12 at timed intervals, or at a predetermined time prior to when cautery tip 24 can be actuated via switch 28, which may be configured as a two-step switch, activated by the hand or via a foot switch. In one preferred embodiment, it is contemplated that a sensor positioned within tubing 40 or the interior of the electrocautery instrument 20 that allows activation of the electrocautery instrument only after the flow of inert gas is established. In this respect, it is contemplated that such sensor will be operative to determine that an out-going stream of inert gas at a pre-determined volume or flow rate will first be met before the electrocautery instrument can be activated.

Optionally, a cooling mechanism as well known by those familiar in the art may be coupled with the gas supply to thus cool the stream of gas emitted therefrom. As such, the cooling mechanism may be situated at any point along the path of the inert gas including cooling of the gas supply unit itself. In addition to extinguishing any small fire, it may also limit the burn injury of the laser or electrocautery device to the point of contact and thereby limit injury to additional tissue.

Optionally, it is contemplated that an oxygen sensor 26 situated close to the tip of the cautery element 12b would be set such that upon meeting or exceeding a predetermined threshold, would send a signal to control unit 14 via link 44b, which in turn may cause gas contained within gas supply 16 to flow through the device. It is likewise contemplated that the flow of inert gas 48 can be selectively modified based upon the concentration of oxygen detected by oxygen sensor 26 such that when a lesser concentration of oxygen is detected, a lesser amount of inert gas 48 is caused to flow through electrocautery device 12. Conversely, to the extent higher levels of oxygen are detected, a correspondingly higher amount of inert gas 48 will be caused to flow through the device and out towards the distal end 12b of the electrocautery instrument.

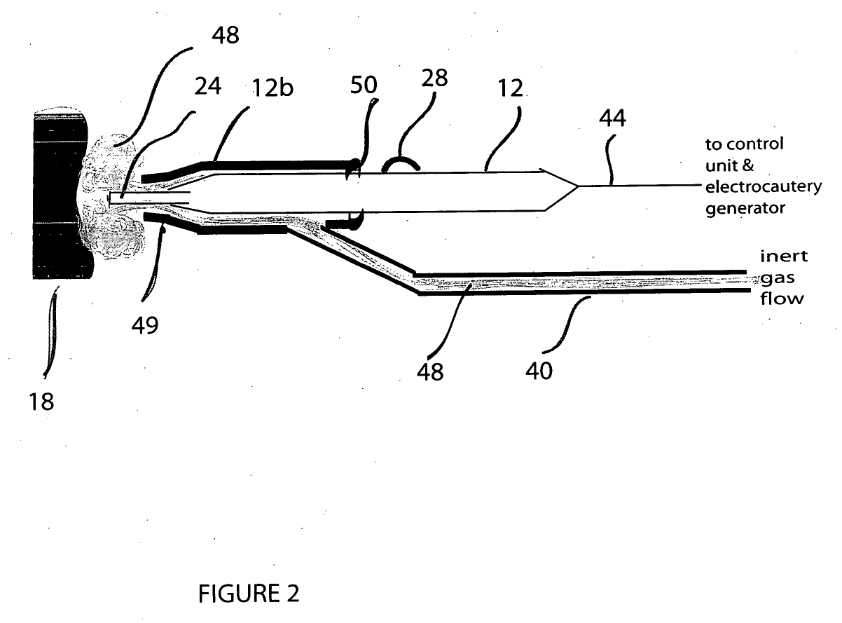

Referring now to FIG. 2, there is shown an alternative embodiment whereby the source of inert gas can be operatively coupled to an existing conventional electrocautery instrument 12. Unlike the embodiment depicted in FIG. 1, there is not provided an internal passageway 20 within the electrocautery instrument 12 through which the inert gas 48 can pass. Rather, the embodiment shown in FIG. 2 is operative to serve as a retrofit whereby the source of inert gas is dispensed through the distal-most end of the electrocautery instrument via the mechanism shown. As illustrated, a housing 49 is positioned axially about the distal most end 12b of the electrocautery instrument 12 such that the electrocautery tip 24 of such electrocautery instrument 12 is allowed to extend therefrom and thus perform its intended purpose to cut through tissue 18. The housing 49, however, radially extends about the electrocautery tip 24 through which the inert gas 48, as supplied by tubing connection 40, will flow about and engulf. In this respect, housing 49 will preferably be provided with an annular collar 50 or other like mechanism that forms an air-tight seal about the distal most end 12b of electrocautery instrument 12. Such arrangement forces the inert gas 48 to be expelled through the distal most end of the housing 49 and thus about the electrocautery tip 24 to thus prevent the same from coming into contact with oxygen to thus prevent the occurrence of a fire.

Advantageously, it is contemplated that the embodiment depicted in FIG. 2 will thus enable the safety mechanisms of the present invention to be readily implemented with existing technology, and not necessarily require specialized electrocautery instrumentation, such as that provided in FIG. 1, to have to be utilized to readily appreciate the advantages of the present invention. As will be readily understood by those skilled in the art, housing 49 may take a variety of shapes and configurations as may be desired to accommodate the various types of electrocautery devices 12 produced by various manufacturers. In this respect, it is contemplated that the housing 49 will be specifically configured that irrespective of any embodiment, there will thus advantageously be channeled a flow of inert gas 48 about the electrocautery tip 24 in whatever manner is necessary to insure that the electrocautery is engulfed about such inert gas and thus prevent the occurrence of an operating room fire. It is likewise contemplated that the configuration of the housing 49 will be such that the same will not interfere with the surgeon's ability to manually manipulate the electrocautery instrument 12, and much less the switching devices, such as 28, necessary to selectively deploy such electrocautery instrumentation.

As will be apparent to those of ordinary skill in the art, the particular combination of parts and steps described and illustrated herein is intended to represent only certain embodiments of the present invention, and is not intended to serve as limitations of alternative devices and methods within the spirit and scope of the invention. In this regard, it is contemplated that the systems of the present invention need not include all of the specific safety features specified herein, namely, the use of an automatic power shut off, free-flowing source of inert gas and/or cut-off of oxygen supply, but may use only one such safety mechanism, or combination of any such mechanisms. Additionally, with respect to the use of an inert gas, it should be emphasized again that the same can be utilized to provide a continuous flow of inert gas through the distal-most end of the electrocautery device, be devised to flow before and after cauterization, selectively provide a flow of inert gas to the extent oxygen concentration levels meet or exceed certain thresholds, or that the flow of such inert gas can increase or decrease based upon the relative concentration of oxygen surrounding the distal end of the electrocautery device. Indeed, it is contemplated that the inert gas may be provided to serve as a flushing mechanism prior to operation of the electrocautery device 12 to thus ensure that prior to any operation of the device 12, that the vicinity surrounding the distal-most end of the electrocautery device, and in particular the cautery tip housed therewithin, are not present in an oxygen enriched environment. In addition, the means of gas delivery to the operative site may be of many different means, including within the housing of the electrocautery device, separately, externally or as an extension of 12b. With regards to the latter design, FIG. 2 demonstrates a separate nozzle 49 added to the distal aspect of the electrocautery element 12b and fitted by a compression fitting 50 or any other related means. This nozzle is further connected to the gas supply via tube 40. Any number of modifications of combination of gas nozzle and cautery/laser application could be contemplated in this regard. Additionally, as per normal gas delivery systems a pressure gauge 53 would be necessary, as well as an alarm system that is set to alert the staff when gas levels are low.

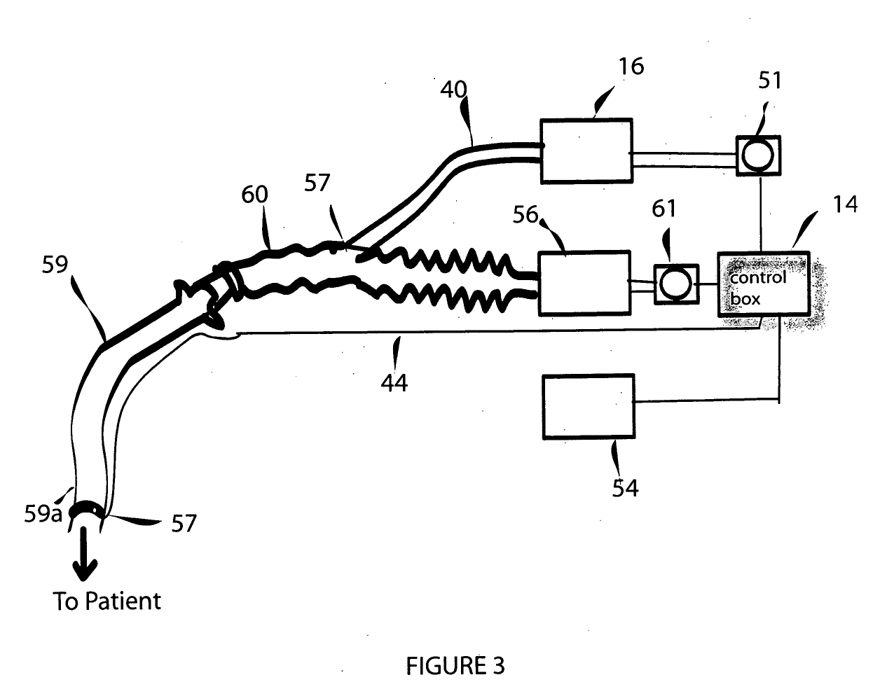

A further embodiment of this comprehensive fire suppression system includes a method to prevent a respiratory apparatus from explosively igniting and fueling an operating room fire. As shown in FIG. 3, a heat sensory device 57, in the form of a thermister or thermocouple would line the distal aspect 59a of the oxygen delivering tube 59, which could be the distal-most end of an endotracheal tube, face mask, nasal cannula, or the like. The thermister would be connected via a link 44 to the control box 14 that is operative to turn the oxygen release valve 61 off when a preset level of temperature is recorded. As such, the ventilation apparatus 56 would not be able to deliver oxygen via its connections 60 to the delivery respiratory device 59 via its lumen 58 to provide further oxidation of the fire. An additional embodiment in this regard would be to fabricate a nonflammable respiratory apparatus from material such as Teflon or an insert of the same or metals to the distal aspect of the delivery apparatus 59a such that the respiratory apparatus may not catch fire in the event of a spark in the presence oxygen.

In addition to selectively controlling the flow of oxygen, it is contemplated that the control unit 14 may further activate an alarm, illustrated as 54. In this respect, once the heat sensory device 57 detects a temperature above a threshold level, the control unit 14 may cause alarm 54 to make an audible signal to thus tell the surgeon and/or anesthesiologist that temperature ranges are exceeding a given safety parameter.

Still further, it is contemplated that the system depicted in FIG. 3 may be readily integrated with the electrocautery systems mentioned above whereby a source of inert gas provided by 16 may be selectively introduced as part of the oxygen provided by ventilation apparatus 56. In this respect, it is contemplated that once heat sensory device 57 generates a signal to control unit 14, the latter may be operative to cause valve 51 to selectively release inert gas 48 from source 16 via tubing 40, and ultimately to tube connections 60 via one way valve 57. Such inert gas will be operative to dilute the concentration of oxygen t or about the distal-most end of the oxygen delivery tube 59 to thus minimize the risk of fire. In a refinement of such system, it is contemplated that the inert gas 48 delivered will preferably be cooled such that when ultimately passed through tubing connection 60 and ultimately to the distal end 59a of oxygen deliver tube 59, such inert gas will be operative to either put the fire out and/or minimize potential trauma to the patient. Indeed, it is contemplated that the inert gas 48 may be operative to flush the tubing system with cool inert gas in the event of activation of the heat sensory device 57.

Additional modifications and improvements of the present invention may also be apparent to those of ordinary skill in the art. Thus, the particular combination of parts and steps described and illustrated herein is intended to represent only certain embodiments of the present invention, and is not intended to serve as limitations of alternative devices and methods within the spirit and scope of the invention. For example, it is contemplated that the inert gas, in addition to being utilized to extinguish or diminish the threat of fire, may also be operative to blow away blood or fluid from the surgical site to thus facilitate the ability to clear the operative area. Accordingly, the present invention should be construed as broadly as possible.

What is claimed is:

1. An electrocautery surgical system for minimizing the outbreak of an operating room fire comprising:
(a) an electrocautery device defined by an elongate housing having proximal and distal ends, said housing further having an electrocautery cautery element disposed therewithin; and
(b) a source of inert gas fluidly coupled to said electrocautery instrument and operative to infuse a flow of inert gas within said housing and through said distal end of said electrocautery device.

2. The system of claim 1 wherein said inert gas is selected from the group consisting of nitrogen, helium, air, carbon dioxide, argon, halon or combinations thereof.

3. The system of claim 2 wherein said invention further comprises a cooling mechanism for cooling said inert gas.

4. The system of claim 1 further comprising an oxygen sensor for detecting the concentration of oxygen at the distal end of said electrocautery device, said oxygen sensor being operative to activate the flow of said inert gas.

5. The system of claim 4 wherein said system further comprises a control unit operatively coupled to said oxygen sensor, electrocautery instrument and said source of inert gas, said control unit being operative to receive signals from said oxygen sensor and regulate the flow of inert gas through said housing of said electrocautery device.

6. The system of claim 1 wherein said electrocautery device is provided with an aperture formed thereon for receiving said inert gas.

7. An electrocautery surgical system for minimizing the outbreak of an operating room fire from electrocautery comprising:
(a) an electrosurgical device selected from the group consisting of an electrocautery device, laser and thermocautery device;
(b) a source of inert gas; and
(c) a housing operative to engage with the operative end of said electrical surgical device, said housing further being coupled to said source of inert gas and operative to infuse a flow of inert gas about the operative end of said electrical surgical instrument when engaged therewith.

8. The system of claim 7 wherein said source of inert gas is selected from the group consisting of nitrogen, helium, air, carbon dioxide, argon, halon or combinations thereof.

9. A respiratory apparatus for use with a ventilation apparatus to minimize the risk of an outbreak of an operating room fire comprising:
(a) an oxygen delivery tube having a distal-most end for delivering oxygen to a patient;
(b) a heat sensory device situated in close proximity to said distal end of said oxygen delivery tube;
(c) a control unit operatively coupled to said heat sensory device, said control unit being operative to adjust the flow of oxygen provided through said oxygen delivery tube in response to signals received from said heat sensory device.

10. The apparatus of claim 9 wherein said control unit is further operative to signal an alarm upon receipt of signals from said heat sensory device indicative of a degree of heat exceeding a predetermined threshold.

11. The apparatus of claim 9 wherein said distal-most end of said oxygen delivery tubing is made from a non flammable material.

12. The apparatus of claim 9 wherein said apparatus further comprises a source of inert gas operatively coupled to said control unit and fluidly connected to said oxygen delivery tube, said control unit being operative to infuse said inert gas from said source of inert gas upon receipt of signals from said heat sensory device corresponding to a temperature exceeding a predetermined threshold.

13. The apparatus of claim 12 further comprises a cooling apparatus for cooling said inert gas provided by said source of inert gas.

14. The apparatus of claim 12 wherein said inert gas comprises a non-toxic flame retardant gas.

15. The apparatus of claim 14 wherein said gas comprises halon.

16. The electrocautery surgical system of claim 7 further comprising a switch, said switch being operative to selectively permit said inert gas to infuse through said housing.

17. The electrocautery surgical system of claim 16 wherein said switch is operative to permit infusion of said inert gas prior to the operation of said electro-surgical device.

18. The electrocautery surgical system of claim 16 wherein said switch is operative to decrease or increase the flow of said gas infused within said housing.

19. The electrocautery system of claim 16 wherein said switch is operative to permit operation of said electrosurgical device upon detection of a flow of inert gas meeting a pre-determined threshold.

20. The electrocautery surgical system of claim 1 further comprising a switch, said switch being operative to selectively permit said inert gas to infuse through said housing of said electrocautery device.

21. The electrocautery surgical system of claim 1 wherein said switch is operative to permit infusion of said inert gas prior to the operation of said electrocautery device.

22. The electrocautery surgical system of claim 1 wherein said switch is operative to decrease or increase the flow of said gas infused within said housing of said electrocautery device.

23. The electrocautery system of claim 1 wherein said switch is operative to permit operation of said electrocautery device upon detection of a flow of inert gas through said housing of said electrocautery device meeting a pre-determined threshold.
